(19) United States
(12) Patent Application Publication
(10) Pub. No.: US 2009/0137628 A1
(43) Pub. Date: May 28, 2009

(54) Formulations of Indanylamines and the Use Thereof as Local Anesthetics and as Medication for Chronic Pain

(75) Inventors: A.K. Gunnar Aberg, Sarasota, FL (US); Keith Johnson, Durham, NC (US)

Correspondence Address: Nields, Lemack & Frame, LLC, 176 E. Main Street, Suite #5, Westborough, MA, 01581, US

(73) Assignee: BRIDGE PHARMA, INC., Sarasota, FL (US)

(21) Appl. No.: 11/991,771

(22) PCT Filed: Sep. 22, 2006
(86) PCT No.: PCT/US2006/037070

§ 371 Date: Jun. 16, 2008

Related U.S. Application Data
(60) Provisional application No. 60/719,904, filed on Sep. 23, 2005.
(60) Provisional application No. 60/839,783, filed on Aug. 24, 2006.

(51) Int. Cl. A61K 31/4458; A61P 25/00

(52) U.S. Cl. 514/319

(57) ABSTRACT

Dermal and mucosal formulations of 2-[2-(N-phenyl-N-2-indanyl)aminoethyl]piperidine, characterized by rapid dermal and mucosal absorption and long duration of therapeutic activity are provided. Also provided are solutions of 2-[2-(N-phenyl-N-2-indanyl)aminoethyl]piperidine for injection, characterized by short onset time and long duration of activity. Formulations containing both 2-[2-(N-phenyl-N-2-indanyl)aminoethyl]piperidine and capsaicin are also provided.

This application claims priority of U.S. Provisional Application No. 60/719,904 filed Sep. 23, 2005 and U.S. Provisional Application No. 60/839,783 filed Aug. 24, 2006, the disclosures of which are incorporated herein by reference.

### BACKGROUND OF THE INVENTION

Local anesthetics are used to prevent expected painful conditions and to treat patients suffering from pain, including neuropathic pain. Local anesthetics are used in both human and veterinary medicine.

The present invention refers to new formulations containing the local anesthetic compound 2-[2-(N-Phenyl-N-2-indanyl)aminoethyl]piperidine (“LAC-34”) and other therapeutic entities, such as for example capsaicin and carefully selected concentrations of various excipients, such as for example solvents, carriers, penetration enhancers, occlusive agents, and emollients. In order to obtain pain-free drug delivery and analgesic activity of long duration, capsaicinoids such as capsaicin may be included in the new formulations described here.

LAC-34 is an extremely potent local anesthetic that also has analgesic effects. Thus, LAC-34 is useful as a local anesthetic and as an analgesic drug. Since LAC-34 was found to have very unusual solubility properties, standard formulations for this drug are not useful and new formulations containing LAC-34 have now been developed. This was particularly the case for dermal formulations of LAC-34.

The mechanism of action of local anesthetic drugs and formulations thereof for the treatment of acute pain, such as for example pain caused by surgery, is a temporary inhibition of nerve conduction in afferent sensory nerves, which is caused by inhibition of sodium channels in said neuronal tissues.

The mechanism of action of drugs and formulations thereof for the treatment of chronic pain, such as for example neuropathic pain, is not just to offer temporary inhibition of afferent nerve conduction in afferent nerves, but also to offer analgesic activity to the patient, which may be achieved by numerous and largely unknown mechanisms.

The indanylamine compound of Formula I has now been found to have such analgesic activities and the formulations of the present invention have been found to facilitate the transport of said chemical entities from the application site to the biophase, where the compounds can exert therapeutic activity.

In combination with certain other compounds that influence the function of sensory nerves, such as for example capsaicin, a useful synergism and a surprising potentiation of the therapeutic benefits has been found with regard to the effects on acute pain and particularly with regard to the effects on chronic pain.

The term “topical anesthesia” is in this document defined as local anesthesia of mucosal membranes, such as for examples those of the eye, the ear, the mouth, the nose, the rectal area and the urogenital tract. The term “dermal anesthesia” is in this document defined as local anesthesia of the skin, including the foreskin. The term “infiltration anesthesia” refers to anesthesia of minor nerves and nerve endings by infiltrating the tissues containing such nerves or nerve endings with a formulation containing a local anesthetic compound. The term “nerve block” in this document refers to the blockade of nerve transmission by administering a formulation of local anesthetic compound close to said nerve. The term “local anesthesia” in this document includes all anesthesia of afferent and efferent nerves and includes “dermal anesthesia”, topical anesthesia” “infiltration anesthesia” and “nerve blocks”

The terms “neuropathic pain” and “neuropathy” refer to a group of chronic painful conditions characterized by pain originating from neural damage. This affects more than 10 million people in the United States alone. This group of patients includes those with a wide variety of primary conditions, which precipitate the debilitating symptoms of thermal and mechanical hyperalgesia that are typical of neuropathic pain.

The terms “analgesic” and “analgesia” as used here refer to relief of pain by drugs that offer analgesic effects, regardless of their mechanism(s) of action.

Although the field of neuropathic pain is large both in terms of patient numbers, suffering, and economic impact on individuals and society, the understanding of neuropathic pain is not complete. The first annual International Conference on the Mechanisms and Treatment of Neuropathic Pain was held in 1998 and in the same year, two publications in the Journal of the American Medical Association focused the interest on the use of the anti-seizure drug gabapentin to treat pain in patients with neuropathic pain.

The various conditions of neuropathic pain have presented clinicians with multiple challenges ranging from difficulties in diagnosis to a lack of effective treatments. When the diagnosis has been made, clinicians typically resort to treating neuropathic pain with old medications that were originally developed for other indications. Due to the lack of effective medications, treatment decisions are often made on a trial and error basis and therapeutic agents, such as tricyclic antidepressants, gabapentin, lidocaine patches, narcotics such as oxycontin, and painkillers such as tramadol are being used. The development of improved experimental models and a better understanding of the pathophysiology of the different neuropathic pain conditions has recently made the rational testing possible of compounds that may be useful for neuropathic pain. Additionally, clinical evaluation of these and existing agents in well-defined patients groups will allow treatment guidelines to further improve. Better knowledge of various types of neuropathic pain will allow the scientific community to select target indications of drugs for neuropathic pain, such as for example neuropathic low back pain, diabetic neuropathic pain, HIV-related neuropathic pain, shingles and various other hyperalgesic and/or neuropathic conditions. Thus, gabapentin and similar compounds are being tried in patients suffering from Neuropathic Low Back Pain (NLBP), while lidocaine patches and capsaicin may offer temporary and more short-acting relief of Diabetic Neuropathic Pain and Shingles. However, and as mentioned above, NSAIDs, narcotics, and even antidepressants are presently used by patients and doctors who desperately try to find medication to decrease the intensity of neuropathic pain. Parenteral injections or dermal application of formulations containing capsaicin or similar agents that may induce degeneration of interneurons in the substantia gelatinosa of the dorsal spinal cord offer promise, particularly if the severe acute pain in association with the drug administration can be eliminated.

Thus, the drug described herein, LAC-34, is useful both as a local anesthetic and as medication for patients suffering from neuropathic pain. When used as a local anesthetic (and particularly as a dermal anesthetic), it is important that the formulation of LAC-34 offers a short onset time of anesthesia. On the other hand, when used as medication for chronic neuropathic pain, the emphasis is on long duration of analgesia rather than fast onset of acute pain relief.

The preparation of effective, efficient formulations containing LAC-34 has proved to be challenging due to the unusual solubility profile of this active moiety. Thus, the free base of LAC-34 has low solubility in water and is therefore not very useful for injection. Several pharmaceutically acceptable salts exist that have improved water solubility, such as for example the di-hydrochloride salt of LAC-34. However, in preclinical studies it has now been found that the free base of LAC-34 offers advantageous dermal anesthesia when compared with various salt forms.

In accordance with the present invention, dermal compositions have now been formulated, such as creams, gels and aerosols containing LAC-34 free base, but also containing various salts of LAC-34, ranging from salt forms with very low water solubility, such as for example the monohydrochloride salt, to salt forms with high water solubility, such as for example the dihydrochloride salt. The new formulations express (a) local anesthetic activities, making them useful as dermal and topical anesthetics and (b) analgesic activities, making them useful in the treatment of chronic pain, in particular for the treatment of neuropathic pain. The dermal formulations typically contain pre-determined concentrations of LAC-34 free base, optionally in combination with one or more solvents, carriers, penetration enhancers, occlusive agents, and/or emollients. The combination of excipients provides means of delivering the drug from highly concentrated (e.g., supersaturated) compositions through skin where the penetration barriers are decreased by the formulations, while the formulations also may repair barrier damage, protect the skin, and hydrate the skin.

The combination of oral or parenteral medication for neuropathic pain with one or more drugs of the present invention can result in a potentiated therapeutic activity. Thus LAC-34 and/or capsaicin dosed dermally or parenterally may be combined with a drug such as for example gabapentin (Neurontine®), phenyloin (Dilantin®) or carbamezepine (Tegretol®) that is dosed orally to the patient.

### SUMMARY OF THE INVENTION

This invention relates to formulations containing LAC-34, to methods of using said formulations as local anesthetics, methods of inducing local, topical or dermal anesthesia, methods of administering the formulations, particularly to a localized region of a patient, and for the treatment of pain and in particular, chronic pain.

The chemical structure of LAC-34 is:

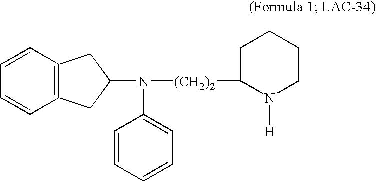

The chemical compound LAC-34 (2-[2-(N-Phenyl-N-2-indanyl)aminoethyl]piperidine) exists as a free base as well as numerous salts. The compound has pharmacological properties that render said compounds to be useful as local anesthetics to inhibit acute pain (U.S. Pat. No. 6,413,987 B1, incorporated herein by reference) and as analgesic medication to prevent and to treat pain, in particular chronic pain, such as neuropathic pain. It achieves short onset time and long duration of local anesthesia, topical anesthesia and dermal anesthesia.

It has now been found that the solubility profile of LAC-34, as the free base, can be effectively reduced by the addition of an anti-solvent, such as water, in an amount sufficient to entice the LAC-34 active agent, upon dermal or topical application, to readily penetrate the skin or mucous membrane of a patient and reach the nerve structure in a sufficient concentration to achieve a therapeutic effect. Surprisingly, the anti-solvent does not just have a dilution effect on the formulation; even small amounts of anti-solvent reduce the solubility of the free base dramatically, such that the formulation approaches saturation or becomes saturated, enhancing the propensity of the active agent to come out of solution and enter the skin or mucous membranes. Indeed, the solubility of the free base in various solvents is so high that the free base is virtually ineffective as a dermal or topical anesthetic agent when dissolved in such solvents in the absence of an anti-solvent. The anti-solvent therefore increases the rate at which the LAC-34 penetrates the skin or mucous membranes of a patient compared to identical formulations but devoid of the anti-solvent.

Formulations for parenteral injections of solutions containing LAC-34, contain said compound preferably as a water-soluble salt, such as for example the dihydrochloride salt in a solution that may also contain preservatives, penetration enhancers and vasoconstrictors.

In combination with other compounds, such as capsaicin, a surprising potentiation of the local anesthetic activity has been found.

In combination with compounds, such as capsaicin, a surprising and very significant improvement of the therapeutic activity in subjects suffering from neuropathic pain is now evident.

Prevention and treatment of (a) acute pain, such as for example pain caused by surgical treatment, including circumcision, vaccination, venopuncture, intravenous cannulation and (b) chronic pain, such as for example neuropathic pain, in particular dermal neuropathic pain, using the formulations of this invention may be achieved by applying said formulations containing LAC-34 on the skin or by injecting solutions of LAC-34 to infiltrate biological tissues in the vicinity of nerves.

The compound LAC-34 can also be used as oral therapy for patients suffering from pain, such as for example neuropathic pain. Conventional oral dose forms may be used and controlled release oral formulations may have advantages over regular tablets or capsules. The oral dose of LAC-34 will have to be titrated for the weight and age of the patient, the severity of the condition and the results that may be expected. Oral doses of between 3 mg and 300 mg may be useful, but lower doses may also be used, particularly in combination with dermal or parenteral formulations containing LAC-34 or a combination of LAC-34 and capsaicinoids.

When used to treat cardiac arrhythmias-atrial or ventricular arrhythmias—LAC-34 can be administered transdermally, parenterally or orally. The doses and the frequency of drug administration depends on the size and age of the patient, the severity of the condition and the results that may be expected. Dermal or topical doses of 10 to 500 mg, intravenous doses of from 1 to 100 mg and oral doses of 5 to 200 mg, administered one to four times daily may be useful for patients suffering from cardiac arrhythmias.

Formulations of LAC-34, intended for dermal and topical administration, preferably contain LAC-34 in its free base form. However, several pharmaceutically acceptable salt forms, such as for example the mesylate or the monohydrochloride and other salts with limited water solubility but suitable solubility in various other excipients, may be used for dermal and mucosal administration of LAC-34, and formulations similar to those disclosed here can also be used for various salt forms of LAC-34 and for the optically active isomers of LAC-34.

The optical isomers of LAC-34 have certain advantages over the racemic compound LAC-34 as disclosed in U.S. Pat. No. 6,413,987. Statements regarding the racemate in this document are in general valid also for the optically active isomers of LAC-34. Thus instead of the racemic mixture LAC-34, the optically active isomers thereof can in most cases be used in the formulations described herein. In some cases, some modifications may be needed of the formulations described here, as obvious to those skilled in the art or as determined using routine laboratory procedures by those skilled in the art.

It was unexpectedly found that LAC-34 exerts analgesic activity in animal models of neuropathic pain and this activity was potentiated by the addition of capsaicin that per se has both algesic and analgesic effects. In addition, the acute and severe pain caused by parenteral or dermal administration of capsaicin is eliminated by administration of a composition, containing LAC-34 as the single therapeutic agent or a formulation containing both LAC-34 and capsaicin. The present invention presents that capsaicin can either be conveniently administered together with LAC-34 in the same formulation, or the two therapeutically active entities can be administered separately.

Thus, the present invention provides formulations for treating humans and animals with (A) dermal and topical anesthetic and parenteral local anesthetic formulations containing LAC-34, (B) dermal and topical analgesic and parenteral analgesic formulations containing LAC-34, (C) dermal and topical anesthetic and parenteral local anesthetic formulations containing both LAC-34 and capsaicin, (D) dermal and topical and parenteral analgesic formulations containing both LAC-34 and capsaicin. The term LAC-34 in this context includes, where appropriate, the optically active isomers of LAC-34, and suitable salts and solvates thereof.

### BRIEF DESCRIPTION OF THE DRAWING

FIG. 1 is a graph of peak area versus concentration of LAC-34.

### DETAILED DESCRIPTION OF THE INVENTION

### Dermal and Topical Formulations of Lac-34 Useful for Dermal and Topical Anesthesia and for Analgesia after Dermal or Topical Application

The dermal and topical formulations of the present invention contain the compound LAC-34 or an optically active isomer thereof in concentrations effective for inducing anesthesia, and particularly in concentrations of about 0.05% to about 20%, preferably 0.1% to 10%. Dermal formulations containing LAC-34 may be applied on the skin one to four times daily, depending on the severity of the disease, the condition of the patient and the effect that is sought. Dermal formulations containing a capsaicinoid such as capsaicin contain the capsaicinoid in concentration between about 0.001% and about 5%, preferably 0.01% to 1.0%. As realized by those skilled in the art, higher or lower concentrations of both LAC-34 and a capsaicinoid may prove to be useful and, under certain circumstances, even preferred. Dermal formulations containing LAC-34 in combination with a selected capsaicinoid, such as for example capsaicin, may be applied on the skin one to four times daily, depending on the severity of the disease the condition of the patient and the effect that is sought.

Solutions for dermal application contain one or more excipients in addition to LAC-34 or an optically active isomer of LAC-34. A partial list of suitable pharmaceutically acceptable excipients is shown in Table 1.

TABLE 1
Examples of excipients, evaluated for their
usefulness in formulations of LAC-34 (including solutions,
gels and creams/lotions)

| Excipient | Description | Function |
|---|---|---|
| Capric/caprylic | Semi-polar | Oil phase for emulsion, |
| triglycerides | oil | emollient |
| Dibutyl adipate | Semi-polar | Solvent, emollient |
| | oil | |
| DMSO | Solvent | Solvent, penetration |
| | | enhancer |
| Ethanol | Volatile | Solvent, penetration |
| | solvent | enhancer |
| Hexylene glycol | Solvent | Solvent |
| Isopropyl | Semi-polar | Oil phase for emulsion, |
| myristate | oil | emollient |
| Isopropanol | Volatile | Solvent, penetration |
| | solvent | enhancer |
| Mineral oil | Oil | Oil phase for emulsion, |
| | | semi-occlusive |
| Pentane | Oil | None, used as model |
| | | propellant |
| Propylene | Solvent | Solvent |
| carbonate | | |
| Propylene glycol | Solvent | Solvent, penetration |
| | | enhancer, humectant |
| Triacetin | Semi-polar | Solvent |
| | oil | |
| Water | Water | Solvent, vehicle base, |
| | | humectant, anti-solvent |

As known by those skilled in the art, practically all chemical entities have limited solubility in one or a few of the key formulation excipients. Surprisingly, this was not the case for LAC-34. This compound was very soluble in all the excipients screened, with the exception of mineral oil, as shown in Table 2 (EXAMPLE 2). This made it highly unlikely to find useful standard dermal and topical formulation for LAC-34.

The addition of an anti-solvent to the LAC-34 formulation was found to efficiently and effectively lower the solubility of the LAC-34 in the excipient, and thereby enhance its ability to penetrate the skin or mucous membranes of a patient. Water is an excellent anti-solvent for LAC-34 in various solvents. The decreased solubility is not merely due to the decreased concentration of the solvents, as is obvious to those skilled in the art of pharmaceutical formulations, from the very significant decrease of the solubility when only small amounts of water are added. Thus, water did not merely dilute the solvents, but water acted as an anti-solvent for LAC-34 in the investigated excipients.

Solvents and penetration enhancers are useful in various formulations when combined with LAC-34, including: capric/caprylic triglycerides, dibutyl adipate, DMSO, ethanol, hexylene glycol, isopropyl myristate, isopropanol, mineral oil, pentane, propylene carbonate, propylene glycol, triacetin, water, decylmethylsulfoxide, N,N-dimethyl acetamide, 2-pyrrolidone, N,N-dimethyl formamide, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, oleic acid, laurocapram (azone), limonene, cineole, diethyl-m-toluamide (deet), sodium dodecylsulfate, trimethyl phosphine oxide, tetrahydrofurfuryl alcohol, glycerol, monolaurate, methyl oleate, and propylene glycol monolaurate. The preferred concentration of solvents in dermal formulation will range from 0.4 percent to 96 percent, and will depend on which additional excipients are being used and the concentration of said additional excipients.

Many penetration enhancers are also surfactants and based on the current findings of physico-chemical properties of LAC-34, the following selected anionic surfactants may pair with LAC-34 in various formulations: sodium dodecylsulfate, dioctyl sodium sulfosuccinate, triethanolamine lauryl sulfate and ammonium lauryl sulfate. The preferred concentration of surfactants in dermal formulation of LAC-34 will range from 0.4 percent to 96 percent, and will depend on which additional excipients are being used and the concentration of said additional excipients.

Those skilled in the art will appreciate that certain non-ionic surfactants may be useful in various dermal formulations. Examples of such surfactants are: glycerol monolaurate, propylene glycol monolaurate, sorbitan monolaurate, sorbitan sesquioleate, polysorbate 20, peg-40 stearate, steareth-20, poloxamer 185. In addition, selected cationic surfactants, such as for example cetyl pyridinium chloride and benzalkonium chloride may also pair with LAC-34. Zwitterionic surfactants, such as for example lecithin may also prove to be useful.

Various “solubilizers” may be used and are generally used to improve the drug in the selected vehicle and/or improve the dermal penetration and/or act as humectants. Examples of “solubilizers” are neutral methacrylic acid esters, polyhydric alcohols such as propylene glycol or polyethylene glycol, surfactants such as sodium lauryl sulphate, vitamin E or combinations of various solubilizers.

Permeation enhancers will improve the dermal penetration and are usually lipophilic solvents, such as for example dimethylsulfoxide, or a surfactant such as for example sodium lauryl sulfate or tween, oleic acid, oleic acid/PG, octyl dimethyl para-amino benzoic acid, a polyhydric alcohol such as propylene glycol or combinations of various penetration enhancers.

Plasticizers may be added to improve the softness of the dermal film and may consist of for example PG, polyethylene glycol, dimethylisosorbide acetyltributyl citrate, triethyl citrate or combinations of various plasticizers.

Humectants can be added to promote the retention of moisture in the skin and examples of humectants are polyhydric alcohols, such as for example butylene glycol, propylene glycol, polyethylene glycol, sorbitol and glycerol, or combinations of various humectants.

Anti-solvents can be added to decrease the solubility of molecules in various solvents and examples of anti-solvents are for example ethylene glycol, diethylene glycol, glycerol, 1,2-propandiol, isopropanol and water. In the case of LAC-34, water was found to be an excellent anti-solvent as shown in Table 4 and there was no need to test any synthetic anti-solvents (EXAMPLE 4) The concentrations of anti-solvents needed will depend on the concentration(s) of the solvent or solvents in the formulation and the efficacy of the selected anti-solvent in the selected solvent(s). Depending on the specific circumstances, the concentrations of the anti-solvent may range from one percent of the concentration of the solvent(s) up to 90 percent of the concentration of a specific solvent when other solvents are present. Most often it is enough to use an anti-solvent in concentrations that range from 1 percent to 50 percent of a solvent, but higher concentrations of the anti-solvent may be needed as obvious to those skilled in the art of making dermal and topical formulations.

Suitable polymers may be included in the formulation to form a stable film on a surface, such as skin, when applied. The film is stable if resistant to rubbing. Preferred film-forming polymers include methacrylic polymers and copolymers as well as acrylic polymers and copolymers, such as for example a copolymer of dimethylamine ethyl methacrylate and a neutral methacrylic acid ester, ammonio methacrylate copolymer type A or type B, methacrylic acid copolymer type A or type B, hydroxypropylcellulose, hydroxyethylcellulose, methyl or ethyl cellulose, cellulose acetate, polyvinyl alcohol and povidone. The porosity of the film is considered important and can be increased by adding watersoluble compounds, such as for example propylene glycol, polyethylene glycol, sodium lauryl sulfate, cetomacrogol or transcutol, or combinations thereof.

Where the formulation is an aerosol, the propellant used may be any pharmaceutically acceptable propellant. Preferred propellants include for example butane, isobutene, propane, dimethylether or other hydrocarbons, dichlorodifluoromethane, trichloro-monofluoromethane, dichloro-fluoroethane, monochloro-difluoromethane, difluoroethane, dichloro-tetrafluoroethane, heptafluoropropane, tetrafluoroethane or other fluorocarbones, or a compressed gas, such as for example carbon dioxide or nitrogen.

Aerosol formulations of the present invention cause low skin irritation-particularly in comparison with patches. Aerosol formulations and aerosol dispensers are easy of use and relatively inexpensive to manufacture.

The aerosol dispenser can be a conventional aerosol can with a conventional metered spray aerosol valve. The pump dispenser can be a conventional bottle or can with a conventional metered spray pump.

The composition is typically applied over a fixed, predetermined area of the skin and is usually from 10 cm² to 25 cm² per actuation. Multiple actuations will be used to cover larger areas, such as for example to patients suffering from shingles or other forms of neuropathic pain.

A dermal or topical formulation of LAC-34 may include a high concentration of DMSO or a similar penetration enhancer. Animal experience have indicated that the vehicle may contain as much as 100% DMSO, although most biological studies were performed with vehicle containing 95% DMSO+5% water or less DMSO

### Suspension Formulations

LAC-34 and its salts, or optically active isomers thereof, could also be prepared as suspensions in dermal formulations. In these formulations, LAC-34 would be present as fine to coarse particles with suitable stabilizers and other excipients. The term “nanosuspensions” refers to colloidal dispersions of sub-micron-size particles of a drug, which are most often stabilized by one or more surfactants in the suspension. Sub-micron size particles of a drug such as LAC-34 may also be dispersed in lipidic carriers. Nanosupensions are of interest for LAC-34 since such suspension may be supersaturated with LAC-34, Such a supersaturated suspension being stable until intentionally destabilized to form saturated or supersaturated dermal formulation. Suspension formulations with particles of LAC-34, its salts or optically active isomers, wherein said particle sizes may range from coarse to submicron size, are encompassed in the present invention.

### Devices

LAC-34 delivery can be facilitated using devices such as a dermal patch or an injector. The patch could deliver LAC-34, or an optically active isomer thereof, to the skin by passive diffusion or by using an active delivery system, such as for example iontophoresis or sonophoresis. Injectors could have a needle or be needless. The doses of LAC-34 used in dermal or transdermal patches will be similar to the doses of lidocaine, presently used in Lidoderm® patches. Due to the low tissue toxicity of LAC-34, higher concentrations, such as 5% to 10% may also prove to be useful in a patch formulation containing LAC-34. Dermal or transdermal patches may be applied one or more times daily. Needless injectors will have obvious advantages, such as for example in selected patients, such as children, and may be used in connection with dermal pain (insect bites, etc) or in preparation for expectedly painful injections with a regular syringe or in preparation for dermal surgical procedures.

In general, the compositions of the present invention are prepared by dissolving the active ingredient, LAC-34, as a racemate or as a single isomer, in either its free base or in a salt form, in a pharmaceutically acceptable solvent, such as DMSO, at room temperature, and adding an anti-solvent, such as water, to the solution. Optional ingredients such as capsaicinoids, permeation enhancers, solubilizers, plasticizers, propellants (in the case of aerosols) may be added before or after the anti-solvent. Specific information for various compositions of formulations are given in the following examples.

### EXAMPLE 1

### Development of Analytical Method

Racemic LAC-34 as a free base (Lot # GLS-L7-163) was used for this study. All solvents were USP, NF, ACS Reagent, or HPLC (acetonitrile) grade. The free base of LAC-34 can exist as a white powder or as a yellowish-brown crystal or oil.

A liquid's capability to solubilize LAC-34 was screened by adding a known mass of LAC-34 to a known volume/mass of liquid. Saturation solubility was measured by adding an excess of drug substance and allowing the suspension to equilibrate while stirring for 2-3 days. A sample of the suspension was filtered though a 0.2 μm PTFE membrane followed by dilution (if required) and HPLC analysis. For several mixed solvent systems that included water, a known mass of drug substance was dissolved in a known mass of solvent(s). This solution was then titrated with water while stirring until the solution became cloudy. All solubility measurements were conducted at room temperature (approximately 21-23° C.).

HPLC analysis was performed with a HP 1050 system (He degasser, quaternary pump, auto-injector, and variable wavelength detector). The column heater was manufactured by Waters. Data collection and analysis was performed using Chemstation® software.

### Results and Discussion

### HPLC Analytical Methodology.

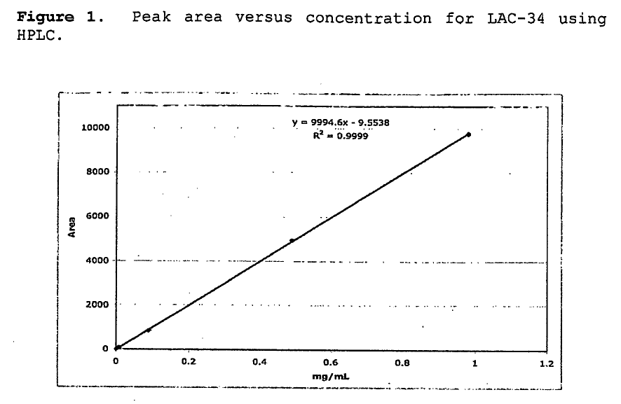

HPLC analytical methodology was developed. Four solutions of LAC-34 (8.1 mcg/mL to 0.98 mg/mL) plus a blank were injected in triplicate. Good linearity for the response is shown in FIG. 1.

The relative standard deviations of peak areas for all LAC-34 solutions were less than 0.5%. The blanks assayed before and after LAC-34 solutions showed no significant drug level, which demonstrated there was no carry-over.

Based on the reproducibility of results over three orders of magnitude in LAC-34 concentrations, no carry-over, and good linearity in the anticipated concentration range, the analytical method was considered acceptable to measure LAC-34 solubility.

### EXAMPLE 2

Various excipients were tested for their capability to dissolve LAC-34, free base. Some results from these screening tests are shown in Table 2.

TABLE 2
Solubility of LAC-34 free base - screening
tests

| | Solubility of LAC-34 (base; |
|---|---|
| Excipient | mg/mL) |
| Polar | |
| Ethanol | >86 |
| Isopropyl alcohol | >79 |
| Propylene glycol | >76 |
| Benzyl alcohol | >100 |
| Propylene carbonate | >78 |
| Hexylene glycol | >75 |
| Semi-polar/non-polar | |
| Dibutyl adipate | >79 |
| Isopropyl myristate | >78 |
| Isopropyl palmitate | 50-100 |
| Mineral oil | <50 |

With the exception of mineral oil, LAC-34 could be dissolved in high concentrations in all the solvents tested. LAC-34 has a yellowish-brown color when it exists as a glass and solutions in Table 2 were yellowish-brown.

When tested at 4° C., 20% LAC-34 in benzyl alcohol was still a solution, 5% LAC-34 in propylene glycol was a gel, 5% LAC-34 in isopropyl myristate was a suspension, 5% LAC-34 in isopropyl palmitate was a solid, 5% LAC-34 in isopropyl alcohol was a suspension and cetyl alcohol was a solid (melting point=49.3° C.).

### EXAMPLE 3

### Tests of Saturation Solubility in Selected Solvents

The saturation solubility of LAC-34 was measured in capric/caprylic triglycerides (CCT), dimethylsulfoxide (DMSO), ethanol, isopropanol, isopropyl myristate (IPM), pentane, propylene glycol (PG), and water. CCT and IPM were selected for further work since they can function as emollients and oily vehicles in creams. DMSO is a solvent and a penetration enhancer. Ethanol and isopropanol are volatile solvents and rapid evaporation of a solvent like ethanol and isopropyl alcohol may be important for minimizing therapeutic onset time. Propylene glycol has multiple functions in dermal formulations, the most important of which are solubilization and penetration enhancement. Some results from the saturation studies with LAC-34 are shown in Table 3.

TABLE 3
Saturation solubility of LAC-34 free base in
key solvents for dermal drug delivery.

| | LAC-34 Solubility, |
|---|---|
| Solvents | mg/mL |
| Capric/caprylic | 195 |
| triglycerides (CCT) | 215 |
| DMSO | >2,000 |
| Ethanol | >526, <2,000 |
| Isopropanol | 184 |
| Isopropyl myristate (IPM) | 149 |
| Propylene glycol (PG) | 0.005 |
| Water | |

All of the solvents, with the exception of water, were able to dissolve large quantities of LAC-34. LAC-34, as the free base, was very soluble also in DMSO. Both IPM and CCT, which are semi-polar emollient oils, had also high solubilization capacity for LAC-34. Propylene glycol (PG), which has multiple actions for dermal drug delivery (see Table 1), was also able to solubilize significant quantities of LAC-34. Ethanol had a very high capacity to dissolve LAC-34 and was never saturated. The value for ethanol in Table 3 was obtained by adding 0.25 g of ethanol to 0.498 g of LAC-34, which rapidly resulted in a homogeneous solution. After observing this phenomenon with ethanol, 0.125 g of isopropanol was added to 0.25 g of LAC-34. Surprisingly, isopropanol was not able to solubilize the drug until the isopropanol level was 0.225 g. This was approximately 526 mg/mL. Since ethanol is very volatile and has a surprisingly high solubilization capacity for LAC-34, having a portion of isopropanol in a dermal formulation for LAC-34 may be useful in creating saturated solutions on the skin. Additional opportunities exist to create super-saturated solutions on the skin by using crystallization inhibitors, such as for example a povidone (Lipp R. selection and use of crystallization inhibitors for matrix-type transdermal drug-delivery systems containing sex steroids. J. Pharm Pharmacol, 1998, 50: 1343-1349), such as for example polyvinyl pyrrolidone (Povidone, Merck Index 12^{th} Ed., 7879).

### EXAMPLE 4

### Tests of Anti-Solvents/Binary Solvent Mixtures

Due to the very high solubility of LAC-34 free base in numerous volatile solvents, it was determined to be necessary to find and add an anti-solvent in order to reach saturation of the solutions of LAC-34. The efficacy of water is an anti-solvent for LAC-34 free base was investigated in the present studies. Table 4 shows the impact of water on solubility in some important solvents.

TABLE 4
Solubility of LAC-34 (in mg/mL) in mixtures of
solvent and water.

| % Water | PG | Ethanol | DMSO |
|---|---|---|---|
| 0 | 149 | >2000 | 215 |
| 1 | 139 | — | — |
| 2.5 | 104 | — | — |
| 5 | 63 | — | ≧100** |
| 10 | 29 | — | — |
| 15 | — | 63* | — |
| 20 | 8.7 | 33* | — |
| 30 | — | 7.0* | — |
| 50 | 0.37 | — | 1.7 |
| 75 | 0.075 | — | — |
| 90 | — | — | 0.45 |

*Titrated from an ethanol/LAC-34 solution with water
**previous tests

As shown in Table 4, water was an excellent anti-solvent for LAC-34 in various solvents. This surprising finding was considered to be important since water is an endogenous molecule found in all mammals and additional toxicity testing of synthetic anti-solvents will therefore not be needed. The decreased solubility is not merely due to the decreased concentration of the solvents, as is obvious to those skilled in the art of pharmaceutical formulations, from the very significant decrease of the solubility when small amounts of water is added. Thus, water did not merely dilute the solvents, but water acted as an anti-solvent for LAC-34 in the investigated excipients.

### EXAMPLE 5

### Tests of Ternary solvent mixtures

Laboratory studies have now demonstrated the compatibility of combinations of various solvents with water as an anti-solvent. Thus, the solubility of 150 mg of LAC-34 free base in a mixture of 1 ml of polyethylene glycol and 1 ml of ethanol was dramatically decreased to 5.5% (w/w) by the presence of only 0.6 ml of the anti-solvent (water).

Suitable dermal formulations of LAC-34 include (1) solutions, (2) gels, (3) creams or lotions, (4) ointments, (5) suppositories (6) aerosols or (7) sprays.

### EXAMPLE 6

### Aqueous and Non-Aqueous Solutions

TABLE 6
Solutions of LAC-34, and the optically active
isomers thereof for dermal and topical applications:

| | % w/w of |
|---|---|
| Component | Formulation |
| RS, R— or S-LAC-34 | 0.1 to 20.0 |
| (base) | |
| Ethanol | 5.0 to 25.0 |
| Propylene glycol | 10.0 to 75.0 |
| Antioxidants¹ | 0.0 to 0.1 |
| Preservatives² | 0.0 to 0.5 |
| Penetration | 0.0 to 50.0 |
| enhancers³ | |
| Water/buffer | q.s. |

¹Examples: BHA, BHT
²Examples: ethyl paraben, propyl paraben, imidurea
³Examples: DMSO, diethylene glycol monoethyl ether, n-decyl methyl sulfoxide, dimethylacetamide, laurocapram (Azone ®), dimethylformamide; sucrose monooleate, N-methyl-2-pyrrolidine (Pharmasolve ®), oleic acid

The solutions will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

Capsaicin (0.01% to 1.0%; W/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin homodihydrocapsaicin can also be used in analogous amounts, as can the partial capsaicin receptor antagonist capsazepine and other compounds that directly increase the inflow of calcium through the neuron membranes or indirectly influence ionic flow through neuron membranes through activation of the vanilloid subtype-1 receptor (such as for example resiniferatoxin),

### EXAMPLE 7

### Methods of Manufacture: Solutions for Dermal and Topical Application

Dissolve LAC-34 in the solvent where it has the highest solubility (e.g., ethanol or isopropyl alcohol). Add additional solvents with preservatives, penetration enhancers, and antioxidants; then mix until well combined. Add water with buffers as needed. Mix until well combined then pack into appropriate container/closure system. The racemic form of LAC-34 or an optically active isomer thereof can be used. The free base or a suitable salt form can be used. The solutions will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### EXAMPLE 8

### Aqueous or Non-Aqueous Gels

A dermal gel formulation of LAC-34 can contain PG, water or buffer, ethanol, a polymer, and optionally another penetration enhancer. Since the amount of water in the formulation may be small, polymers that can gel non-aqueous system such as hydroxypropylcellulose (HPC) or hydroxyethylcellulose (HEC) can be used. In experiments with HEC, this excipient was found not only to ably gel the systems, but surprisingly, HEC was also found to be able to stabilize supersaturated solutions of LAC-34 on the skin's surface. As persons skilled in the art will realize, other topically acceptable gelling agents and may also prove to be useful in gels containing LAC-34.

TABLE 7
Gels containing LAC-34 base for dermal
applications:

| | % w/w of |
|---|---|
| Component | Formulation |
| RS—, R— or S-LAC- | 0.1 to 20.0 |
| 34 | |
| Ethanol | 5.0 to 25.0 |
| Propylene glycol | 50.0 to 75.0 |
| Polymer¹ | 0.5 to 3.0 |
| Antioxidants² | 0.0 to 0.3 |
| Preservatives³ | 0.0 to 0.5 |
| Penetration | 0.0 to 20.0 |
| enhancers⁴ | |
| Water/buffer | q.s. |

¹Examples: Hydroxypropylcellulose, hydroxyethylcellulose, carbomer
²Examples: BHA, BHT
³Examples: Methyl paraben, propyl paraben, imidurea
⁴Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid

The gels will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin homodihydrocapsaicin can also be used in analogous amounts, as can the partial capsaicin receptor antagonist capsazepine and other compounds that directly or indirectly influence ionic flow in neuron membranes.

### EXAMPLE 9

### Methods of Manufacture: Gels

Dissolve LAC-34 in the solvent where it has the highest solubility (e.g., ethanol or isopropyl alcohol). Add additional solvents with preservatives, penetration enhancers, and antioxidants then mix until well combined.

Disperse polymer under appropriate conditions recommended by the manufacturer. Add water with buffers (if required) and neutralizing bases as needed. Mix until well combined and thickened then pack into appropriate container/closure system.

The racemic form of LAC-34 or an optically active isomer thereof can be used. The free base or a suitable salt form can be used. The gels will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### EXAMPLE 10

### Emollient Creams and Lotions

The surprisingly high solubility of LAC-34 in emollient oils was exploited to develop elegant cream formulations. When the oil-phase was approximately 55%, a 10% LAC-34 cream could be made with IPM or CCT. A penetration enhancer could also be included.

TABLE 8
Creams containing LAC-34 base for dermal
applications:

| | % w/w of |
|---|---|
| Component | Formulation |
| RS—, R— or S-LAC-34 | 0.1 to 20.0 |
| Capric/Caprylic | 0.0 to 50.0 |
| Triglycerides | |
| Isopropyl myristate | 0.0 to 50.0 |
| Propylene glycol | 0.0 to 25.0 |
| Surfactant | 1.0 to 5.0 |
| Polymer¹ | 0.5 to 3.0 |
| Antioxidants² | 0.0 to 0.3 |
| Preservatives³ | 0.0 to 0.5 |
| Penetration enhancers⁴ | 0.0 to 30.0 |
| Water/buffer | q.s. |

¹Examples: Hydroxypropylcellulose, hydroxyethylcellulose, carbomer, xanthan gum
²Examples: BHA, BHT
³Examples: Methyl paraben, propyl paraben, imidurea
⁴Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid

The emollient creams or lotions will preferably contain from 0.1 percent to 30 percent of 2-[(2-(N-phenyl-N-2-indanyl)aminoethyl]piperidine, or an optically active isomer thereof, in its free base form or as a pharmaceutically acceptable salt.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

### EXAMPLE 11

### Methods of Manufacture: Creams

Combine oils, semi-polar oils, antioxidants, penetration enhancers, and surfactants and heat to 60-75° C. (Phase A). Combine polar solvents, preservatives, water, buffer salts, and polymer and heat to 60-75° C. (Phase B) Dissolve LAC-34 in Phase A then immediately

add Phase A to Phase B with rapid stirring. High-shear mixing for a specified time may also be required. Cool the mixture while stirring to 30-50° C. If the polymer requires a basic substance to thicken (e.g., sodium hydroxide or triethanolamine), add the base. Once product is sufficiently thickened and cool (≦35° C.), stop stirring and begin packing the product into the appropriate container closure system.

The racemic form of LAC-34 or an optically active isomer thereof can be used. The free base or a suitable salt form can be used. The emollient creams or lotions will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### EXAMPLE 12

### Ointments for Dermal, Rectal and Vaginal Application

While the unexpectedly high solubility of LAC-34 base in practically all excipients (not water) complicated the formulation of dermal solutions, gels and cream containing LAC-34, the high solubility of LAC-34 in “oily” excipients like IPM and mineral oil can also bee seen as an advantage since such excipients tend to be non-irritating and may be suited to an emollient cream formulation for extended use and for ointments (i.e., hemorrhoids, shingles, etc.).

TABLE 9
Ointments (and non-irritant creams) containing LAC-
34 for dermal and rectal use

| | % w/w of |
|---|---|
| Component | Formulation |
| RS—, R—, or S-LAC- | 1.0 to 20.0 |
| 34 | |
| Propylene glycol | 2.5 to 10.0 |
| Microcrystalline | 5.0 to 30.0 |
| wax | |
| White wax (bees | 0.0 to 20.0 |
| wax) | |
| Surfactants | 0.5 to 5.0 |
| Antioxidants¹ | 0.0 to 0.1 |
| Penetration | 0.0 to 20.0 |
| enhancer² | |
| Mineral oil | q.s. |

¹Examples: BHA, BHT
²Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid

The ointments and non-irritant creams will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin homodihydrocapsaicin can also be used in analogous amounts, as can the partial capsaicin receptor antagonist capsazepine and other compounds that directly or indirectly influence ionic flow in neuron membranes.

### EXAMPLE 13

### Methods of Manufacture: Ointments

Combine waxes, oils, and semi-polar oils, and antioxidants and heat to 60-75° C. (Phase A). Combine LAC-34 with solvent (e.g., propylene glycol) and penetration enhancer (if used) and heat to 60-75° C. (Phase B). Add Phase B to Phase A and mix under high shear while cooling. Once ointment thickens, stop high shear and mix with low shear until product is sufficiently cool (≦35° C.), stop stirring and begin packing the product into the appropriate container closure system.

The racemic form of LAC-34 or an optically active isomer thereof can be used. The free base or a suitable salt form can be used.

The ointments and non-irritant creams will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### EXAMPLE 14

### Suppositories for Rectal or Vaginal Applications

Suppositories are useful for the treatment of vaginal and rectal pain, particularly hemorrhoidal pain.

TABLE 10
Suppository formulations containing LAC-34
base for rectal or vaginal applications.

| | % w/w of |
|---|---|
| Component | Formulation |
| RS—, R— or S-LAC-34 | 0.1 to 20.0 |
| Antioxidants¹ | 0.0 to 0.3 |
| Preservatives² | 0.0 to 0.5 |
| Penetration enhancers³ | 0.0 to 10.0 |
| Suppository maxtrix⁴ | q.s. |

¹Examples: BHA, BHT
²Examples: Methyl paraben, propyl paraben, imidurea
³Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid
⁴Polyethylene glycol (molecular weights 200 to 20,000), hydrogenated vegetable oil (e.g., Wecobee ® S, Wecobee ® FS

The suppositories will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

### EXAMPLE 15

### Methods of Manufacture: Suppositories

Melt suppository matrix material (i.e., polyethylene glycol or partially hydrogenated vegetable oil) then dissolve LAC-34 in the melt. Fill melt into mold, cool, and un-mold. Pack suppositories in appropriate container/closure system.

The racemic form of LAC-34 or an optically active isomer thereof can be used in concentrations from about 0.1 percent to about 20 percent. The free base or a suitable salt form can be used.

The suppositories will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### EXAMPLE 16

### Aerosol Sprays (Pump or Propellant Driven)

In addition to the solubility experiments shown in Table 3, the solubility of LAC-34 (base) was also investigated using pentane since this is an example of low molecular weight hydrocarbons, commonly used to model a GRAS aerosol propellant such as butane. The result is shown below. The surprisingly very high solubility of LAC-34 in pentane indicates that hydrocarbon aerosol propellants such as butane will be useful for drug delivery to the skin by aerosol.

TABLE 11
Saturation solubility of LAC-34 in pentane.

| Solvent | LAC-34 Solubility, mg/ |
|---|---|
| Pentane | 150* |

*Pentane evaporates rapidly and is difficult to weigh. This value, therefore, is not exact.

Thus, since LAC-34 is very soluble in both volatile solvents and low molecular weight hydrocarbons, an aerosol formulation of LAC-34 is also possible. The aerosol can contain a volatile solvent or propellant along with a non-volatile solvent plus one or more penetration enhancers. Numerous propellants are known to those skilled in the art and can be used with LAC-34 in aerosol sprays. When ethanol is the volatile solvent, a pump spray will be required to create an aerosol. When butane is used, the container/closure system can be pressurized and only a metering valve is required to create a useful aerosol administration system.

TABLE 12
Examples of aerosol formulations of LAC-34
(propellant driven or pump-driven)

| | % w/w of |
|---|---|
| Component | Formulation |
| RS—, R— or S-LAC-34 | 0.2 to 20.0 |
| Propylene glycol | 0.0 to 40.0 |
| Surfactant | 0.05 to 5.0 |
| Polymer¹ | 0.0 to 3.0 |
| Antioxidants² | 0.0 to 0.3 |
| Preservatives³ | 0.0 to 0.5 |
| Propellants⁴ | 0.0 to 40.0 |
| Penetration enhancers⁵ | 0.0 to 50.0 |
| Water/buffer | q.s. |

¹Examples: Hydroxypropylcellulose, hydroxyethylcellulose, carbomer
²Examples: BHA, BHT
³Examples: Methyl paraben, propyl paraben, imidurea
⁴Examples: Butane, isobutane, propane, hydrofluoralkane (134a, 227)
⁵Examples: DMSO, diethylene glycol monoethyl ether, n-decyl methyl sulfoxide, dimethylacetamide, laurocapram (Azone ®), dimethylformamide, sucrose monooleate, N-methyl-2-pyrrolidine (Pharmasolve ®), and oleic acid

The aerosols will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

Capsaicin (0.01% to 1.0%; w/w) can be included in these formulations to obtain prolonged relief from pain and in particular from neuropathic pain. The compound LAC-34 will inhibit or decrease the initial pain caused by capsaicin. Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

### EXAMPLE 17

### Methods of Manufacture: Aerosols

If the product is a pump spray, prepare solution as described in the solutions/gels section and fill into the appropriate container/closure system with spray pump.

If the product is a propellant-driven spray, dissolve LAC-34 in liquefied propellant and add appropriate solvents, penetration enhancers, and surfactants/polymers. Mix until homogeneous then fill under pressure into the appropriate container/closure system. Alternatively, LAC-34 plus solvents, surfactants/polymers, and penetration enhancers can be combined at room temperature/pressure then filled into an aerosol canister. After crimping the metering valve in place, the propellant can be pressure-filled into the container.

Alternatively, the acrylic polymer or copolymer is dissolved in the chosen vehicle; the active ingredient together with the permeation enhancer is dissolved in the solution, the plasticizer and remaining ingredients are added, the cans are filled, the liquefied propellant is added and the metering valve is crimped in place.

The racemic form of LAC-34 or an optically active isomer thereof can be used for aerosols. The free base or a suitable salt form can be used.

The aerosols will preferably contain from 0.1 percent to 30 percent of 2-[2-(N-phenyl-N-2-indanyl)aminoethyl]piperidine, or an optically active isomer thereof, in its free base form or as a pharmaceutically acceptable salt.

### EXAMPLE 18

### A. Parenteral Formulations for LAC-34, Useful for Local Anesthesia and for Analgesia after Injection

As a local anesthetic, LAC-34 is significantly more active than lidocaine. Thus, the affinity of LAC-34 and lidocaine for the Na⁺ channel (site 2) in the rat cerebral cortex was determined and the inhibitory concentrations of the two local anesthetics were determined. Membrane homogenates of cerebral cortex (250 μg protein) were incubated, in the absence or presence of the test compound, for 60 min at 22° C. with 10 nM [³H]batrachotoxin. Nonspecific binding was determined in the presence of 300 μM veratridine.

IC50-values for inhibition of batrachotoxinin binding were 2.7E-7M and 4.7E-5M for LAC-34 and lidocaine, respectively, which demonstrates that LAC-34 is significantly more effective as a local anesthetic than lidocaine. It is concluded that LAC-34 may be useful in lower concentration than lidocaine or, if used in concentrations similar to lidocaine, the anesthetic depth and the anesthetic duration will be more advantageous for LAC-34 than for lidocaine.

Formulations for injection will preferably contain a watersoluble salt of LAC-34, such as for example the LAC-34 dihydrochloride salt. It was found that this salt is highly water soluble. This salt also binds molecular water.

Surprisingly, and contrary to lidocaine, LAC-34 was found not to cause local vasodilatation at the dermal injection site. It is assumed that the surprisingly long duration of local anesthetic activity of LAC-34 may at least in part be due to the lack of vasodilating effects of this compound. It was, however found that the long duration of local anesthetic activity could be further prolonged by incorporation of a vasoconstrictor, such as epinephrine in concentrations usually used for epinephrine in combinations with local anesthetics, such as lidocaine, and well known to those skilled in the art of medicine.

Since local anesthetics for injection should preferably be water soluble, the free base of LAC-34 is less favored than water-soluble salts, such as for example the dihydrochloride salt of LAC-34. Excipients included in a solution for injections of LAC-34×2HCl are shown in Table 13.

TABLE 13
Formulations of LAC-34 for injections

| | % w/w of |
|---|---|
| Component | Formulation |
| RS—, R— or S-LAC-34 × | 0.1 to 10.0 |
| 2HCl | |
| Chelating agents¹ | 0.0 to 0.3 |
| Antioxidants² | 0.0 to 0.3 |
| Preservatives³ | 0.0 to 0.5 |
| Vasoconstrictors⁴ | 0.00 to 0.05 |
| Water/buffer | q.s. |

¹Examples: Disodium edetate, citric acid
²Examples: BHA, BHT, ascorbic acid
³Examples: Methyl paraben, propyl paraben, imidurea
⁴Examples: Epinephrine, phenylephrine

The solutions for parenteral use will preferably contain from 0.1 percent to 30 percent of 2-[2-(N-phenyl-N-2-indanyl)aminoethyl]piperidine, or an optically active isomer thereof, preferably as a water-soluble, pharmaceutically acceptable salt.

Capsaicin (0.01% to 1.0%; w/w) can be included in these formulations to obtain prolonged relief from pain and in particular from neuropathic pain. The compound LAC-34 will inhibit or decrease the initial pain caused by capsaicin.

Other capsaicinoids, such as dihydrocapsaicin, nordihydrocapsaicin, homocapsaicin and homodihydrocapsaicin can also be used in analogous amounts, as can the partial capsaicin receptor antagonist capsazepine and other compounds that directly or indirectly influence ionic flow in neuron membranes.

### EXAMPLE 19

### Methods of Manufacture: Parenteral Solutions

Combine ingredients, including active, in an appropriate vessel and mix until homogeneous. Aseptically filter and fill the solution into the appropriate container/closure system. Terminal sterilization is also an option in the drug product preparation if required.

The racemic form of LAC-34 or an optically active isomer thereof can be used. The free base or (preferably) a water-soluble salt form can be used.

The solutions for parenteral use will preferably contain from 0.1 percent to 30 percent of 2-[2-(N-phenyl-N-2-indanyl)aminoethyl]piperidine, or an optically active isomer thereof, preferably as a water-soluble, pharmaceutically acceptable salt.

### EXAMPLE 20

### Pharmacological Comparison Between Dermal Anesthetic Activity of the Free Base and Various Salt Forms

The purpose of this study was to determine the degree to which various salt forms of LAC-34 contribute to the local dermal anesthetic effect of the compound, following dermal application to guinea pigs.

Four groups of six male guinea pigs received a dose of 2.0 ml of a 5.0% solution of four test articles (LAC-34 base, LAC-34 mono-HCl, LAC-34-di HCl and LAC-34 mesylate) that had been applied onto a two inch square, 4 layer thick gauze pad on depilated dosal skin of each guinea pig. Similarly, one group of guinea pigs received the vehicle, which consisted of DMSO/water (90 percent/10 percent, v/v) and served as the control group. The gauze was occluded and the guinea pigs were wrapped for 30 min. Animals were assessed for initial pain reaction (anesthetic depth) at 15 minute intervals up to 3 hours and at 24 hours. An algesimeter, set at 10 g, was employed to assess the onset and depth of anesthesia using the pinprick method causing a twitch response or non-response. The “Average Anesthetic Score” was defined as number of probings not producing a dermal twitch response. Thus, a situation where there were no dermal twitch responses to six probings represent a maximal anesthetic effect and is denoted as an Average Anesthetic Score of 6.

### RESULTS

The Average Anesthetic Scores after patch removal are shown in Table 13 (vales have been claculated from Table 1 in the original Report.)

TABLE 13
Average Anesthetic Scores after patch removal.

| | Time after Patch | | | | |
|---|---|---|---|---|---|
| Treatment | Removal (min) | | | | |
| Group | 15 | 30 | 60 | 90 | 180 |
| LAC-34 Free Base | 4.8 | 4.8 | 4.4 | 3.3 | 2.2 |
| LAC-34 Mono-HCl | 0.2 | 0.8 | 1.3 | 0.7 | 0.7 |
| LAC-34 Di-HCl | 1.0 | 1.0 | 2.3 | 0.8 | 0.2 |
| LAC-34 Mesylate | 0.7 | 0.7 | 1.8 | 1.5 | 0.2 |
| Vehicle | 0.0 | 0.0 | 0.0 | 0.0 | 0.0 |

The results shown in the table 13, demonstrate a favorable effect LAC-34 base when compared with the salt forms tested here. The duration of anesthesia was longer for the free bas than for any of the salts and the maximal depth of anesthesia (4.8 for LAC-34 base) was more favorable than for any of the salt forms (2.3 for LAC-34 dihydrochloride)

### CONCLUSION

The dermal anesthetic activity of LAC-34 free base was significantly more pronounced that that of any of the LAC-34 salt forms.

When used to treat pain, LAC-34 can be administered dermally, topically, parenterally or orally. The doses and the frequency of the drug administration depend on the size and age of the patient, the severity of the condition, the administration form, and the results that may be expected. Dermal or topical doses of 10 to 500 mg, intravenous doses of from 1 to 100 mg and oral doses of 5 to 200 mg administered one to four times daily may be useful for patients from suffering from these conditions.

### EQUIVALENTS

Those skilled in the art will realize that liposome formulations containing LAC-34 and the isomers thereof may be clinically useful. There is a wide variety of liposomes available and using no more than routine experimentation, those skilled in art may develop clinically useful formulations containing LAC-34 and suitable liposome suspensions. All equivalents are intended to be encompassed in the scope of the present invention.

All doses indicated in this document refer to human doses. For use in animals, the doses may be the same as human doses or may be lower or higher, depending on the species and the reason for the treatment. The animal caretaker or a veterinarian will be able to determine the useful dose and concentration that is preferred to specific animals.

Doses higher or lower than indicated here may be useful in humans or in animals and all useful dose levels are intended to be encompassed in the scope of this invention.

What is claimed is:

1. A dermal formulation for inducing relief of pain or for preventing pain, comprising a therapeutically effective amount of 2-[2-(N-phenyl-N-2-indanyl)aminoethyl]piperidine, or an optically active isomer thereof, or a pharmaceutically acceptable salt thereof, dissolved in one or more solvents in which said piperidine is soluble, together with an anti-solvent in an amount sufficient to reduce the solubility of said 2-[(2-(N-phenyl-N-2-indanyl)aminoethyl]piperidine in said one or more solvents such that upon application of said formulation to the skin of a patient, said solvent evaporates to form a saturated solution of said 2-[2-(N-Phenyl-N-2-indanyl)aminoethyl]piperidine facilitating the penetration of said 2-[2-(N-phenyl-N-2-indanyl)aminoethyl]piperidine into the skin.

2. A formulation according to claim 1 that contains from 0.1 percent to 30 percent of 2-[2-(N-phenyl-N-2-indanyl)aminoethyl]piperidine, or an optically active isomer thereof, in its free base form.

3. A formulation according to claim 1 that contains from 0.1 percent to 30 percent of 2-[2-(N-phenyl-N-2-indanyl)aminoethyl]piperidine, or an optically active isomer thereof, as a pharmaceutically acceptable salt.

4. A formulation according to claims 1 or 2, wherein the said one or more solvents is a member selected from the group consisting of ethanol, dimethylsulfoxide, isopropanol, propylene carbonate, propylene glycol, decylmethylsulfoxide, N,N-dimethyl acetamide, 2-pyrrolidone, N,N-dimethyl formamide, 1-methyl-2-pyrrolidone, 5-methyl-2-pyrrolidone, 1,5-methyl-2-pyrrolidone, 1-ethyl-2-pyrrolidone, 2-pyrrolidone-5-carboxylic acid, oleic acid, laurocapram (azone), limonene, cineole, diethyl-m-toluamide (deet), sodium dodecylsulfate, trimethyl phosphine oxide, tetrahydrofurfuryl alcohol, glycerol, monolaurate, methyl oleate, and propylene glycol monolaurate, and mixtures thereof.

5. A formulation according to claims 1 or 2, further comprising one or more non-ionic surfactants selected from the group consisting of dimethylsulfoxide, ethanol, isopropanol, propylene glycol, glycerol monolaurate, propylene glycol monolaurate, sorbitan monolaurate, sorbitan sesquioleate, polysorbate 20, peg-40 stearate, steareth-20, poloxamer 185.

6. A formulation according to claim 1 that contains dimethylsulfoxide in a concentration ranging from 1 percent to 100 percent.

7. A formulation according to claim 1, where the formulation is a solution, cream, ointment, lotion, spray, aerosol, foam or paste.

8. A formulation according to claim 1, wherein said anti-solvent is water.

9. A formulation according to claim 1 that is delivered to the skin by the use of a vehicle or device suitable for dermal or epidermal delivery.

10. A formulation according to claim 1, further comprising capsaicin in a concentration from 0.001 percent to 5 percent.

11. A formulation according to claim 9 in the form of a solution, cream, ointment, lotion, gel, spray, aerosol, foam, or paste.

12. A pharmaceutical composition for induction of local anesthesia, comprising as an active agent a local anesthetic inducing effective amount of 2-[2-(N-phenyl-N-2-indanyl)aminoethyl]piperidine, or an optically active isomer thereof, or a pharmaceutically acceptable salt thereof, dissolved in a pharmaceutically acceptable solvent, and further comprising an anti-solvent in an amount sufficient to reduce the solubility of said 2-[2-(N-phenyl-N-2-indanyl)aminoethyl]piperidine in said solvent such that upon application of said composition to the skin of a patient, said solvent evaporates to form a saturated solution of said 2-[2-(N-Phenyl-N-2-indanyl)aminoethyl]piperidine, facilitating the penetration of said 2-[2-(N-phenyl-N-2-indanyl)aminoethyl]piperidine into the skin.

13. The pharmaceutical composition of claim 12, wherein said anti-solvent is water.

14. The pharmaceutical composition of claim 12, wherein said piperidine is in the form of its free base.

15. A method of increasing the rate of penetration of 2-[2-(N-phenyl-N-2-indanyl)aminoethyl]piperidine through the skin of a patient, comprising formulating a composition of said piperidine in a pharmaceutically acceptable solvent with an anti-solvent in an amount sufficient to reduce the solubility of said piperidine in said solvent, and applying said composition to said skin.

16. The method of claim 15, wherein said anti-solvent is water.

17. The method of claim 15, wherein said piperidine is in the form of its free base.

18. The method of claim 15, wherein said piperidine is in the form of a pharmaceutically acceptable salt.

19. The method of claim 15, wherein said piperidine is in the form of its dihydrochloride salt.

20. The method of claim 15, wherein said composition further comprises capsaicin.
